(19) United States
(12) Patent Application Publication
(10) Pub. No.: US 2013/0281821 A1
(43) Pub. Date: Oct. 24, 2013

(54) INTRAOPERATIVE CAMERA CALIBRATION FOR ENDOSCOPIC SURGERY

(71) Applicants: Xin Liu, Scarsdale, NY (US); Kongkuo Lu, Sugar Land, TX (US); Sheng Xu, Rockville, MD (US)

(72) Inventors: Xin Liu, Scarsdale, NY (US); Kongkuo Lu, Sugar Land, TX (US); Sheng Xu, Rockville, MD (US)

(73) Assignee: KONINKLIJKE PHILIPS ELECTRONICS N.V., Eindhoven (NL)

(21) Appl. No.: 13/978,167

(22) PCT Filed: Jan. 3, 2012
(86) PCT No.: PCT/IB2012/050024

§ 371 Date: Jul. 3, 2013

Related U.S. Application Data
(60) Provisional application No. 61/432,298, filed on Jan. 13, 2011.

(51) Int. Cl. A61B 1/00; A61B 1/012; A61B 1/267; A61B 1/04

(52) U.S. Cl. CPC A61B 1/00158; A61B 1/04; A61B 1/0125; A61B 1/00009; A61B 1/2676; A61B 1/0005
USPC 600/409

(57) ABSTRACT

A surgical navigation system employs an endoscope (30) and an imaging unit (80). The endoscope (30) include an electromagnetic tracker (40) within a working channel of endoscope (30) for generating electromagnetic sensing signals indicative of one or more poses of the endoscope (30) within an anatomical region, and an endoscopic camera (50) within an imaging channel of the endoscope (30) for generating endoscopic images of the anatomical region. The imaging unit (80) executes an intraoperative calibration of the electromagnetic tracker (40) and the endoscopic camera (50) as a function of an image registration between the preoperative scan image of a calibration site within the anatomical region and one or more endoscopic images of the calibration site within the anatomical region.

The present invention generally relates to a real-time tracking of a surgical tool within an anatomical region of a body based on a preoperative scan image and endoscopic images of the anatomical region. The present invention specifically relates to a computation of an offset transformation matrix between an endoscopic camera and an electromagnetic (“EM”) tracker using the preoperative scan image and one or more endoscopic images of the anatomical region.

EM guided endoscopy has been recognized as a valuable tool for many lung applications. The advantage of this technology over conventional endoscopy is based on a real-time connection to a three-dimensional (“3D”) roadmap of the lung while the interventional procedure is being performed. This connection requires a tracking of a tip of an endoscope in a global coordinate system to thereby associate endoscopic images of the lung with a preoperative scan image of the lung (e.g., a computed tomography image, a magnetic resonance image, an X-ray image, a three-dimensional ultrasound image, etc.). The fused images are displayed to enable the surgeon to visually navigate the endoscope to a surgical site within the lung.

A key requirement of this image integration is an endoscopic calibration involving a determination of a position and an orientation of an EM tracker externally mounted to the endoscope with respect to a coordinate system of an endoscopic camera disposed within a camera channel of the endoscope. The results of this endoscopic calibration take the form of six (6) offset constants: three (3) for rotation and three (3) for translation. The goal of the endoscopic calibration in an interventional endoscopic procedure is to dynamically determine the pose of the endoscopic camera relative to the preoperative scan image based on the EM readings of the attached EM tracker.

Generally speaking, calibration parameters have been obtained in the art by using an EM-tracked endoscope to image an EM-tracked lung phantom of a particular calibration pattern that has known geometric properties. However, a phantom based endoscopic calibration involves a cumbersome engineering procedure. In one known endoscopic calibration, although a desired transformation of the endoscopic calibration is between a camera coordinate system and an EM tracker coordinate system, an array of calibration procedures are in fact needed between an endoscope, the EM tracker externally and rigidly attached to the endoscope, an EM field generator, the calibration phantom and a reference tracker. For example, the needed calibration procedures include a calibration of the EM tracker coordinate system and the reference tracker, a calibration between the calibration phantom and the reference tracker, and a calibration between the endoscopic camera and the calibration phantom to thereby arrive at the destination calibration between the camera coordinate system and the EM tracker coordinate system.

In addition, the data acquisition protocol required in collecting the calibration data is usually from a calibration phantom with a checker-board pattern. This makes the calibration impractical to be an intraoperative calibration procedure of the endoscopic application. However, an intraoperative calibration is preferred under circumstances whereby (1) intrinsic camera and distortion parameters are fixed and determined through a preoperative calibration process and (2) extrinsic camera parameters (e.g., a transformation between the coordinates of the EM tracker and the endoscopic camera) are not fixed and will change across different endoscopic applications. This change may due to the reality that the EM tracker may not be bundled permanently to the tip of the endoscope due to a variety of reasons. For example, the EM tracker may be inserted inside the working channel of the endoscope at the initial phase of the endoscopic application, removed from the working channel after the endoscope reaches the target site within the anatomical region, and replaced with a surgical instrument (e.g., a biopsy needle or forceps) for subsequent interventions.

Moreover, intraoperative calibration procedures as known in the art still utilize a calibration phantom.

The present invention provides an endoscopic calibration approach that quickly and accurately computes the desired extrinsic parameter to thereby achieve the real-time data fusion between a preoperative scan image (e.g., a CT image) of an anatomical region and endoscopic images of the anatomical region. Specifically, the endoscopic calibration method of the present invention excludes any involvement with any phantom. Instead, the endoscopic calibration method of the present invention utilizes both preoperative scan data and endoscopic video data from a patient to perform an image-based registration that yields the transformation from the preoperative scan coordinates to the endoscopic camera coordinates, which may be utilized with other known transformation matrixes to derive the desired calibration transformation matrix.

One form of the present invention is a surgical navigation system employing an endoscope and an imaging unit. The endoscope includes an electromagnetic tracker within a working channel of the endoscope for generating electromagnetic sensing signals indicative of one or more poses of the endoscope within an anatomical region, and an endoscopic camera within an imaging channel of the endoscope for generating endoscopic images of the anatomical region. In operation, the imaging unit executes an intraoperative calibration of the electromagnetic tracker and the endoscopic camera as a function of an image registration between the preoperative scan image of a calibration site within the anatomical region and one or more endoscopic images of the calibration site within the anatomical region.

In a second form of the present invention, the surgical navigation system further employs an electromagnetic tracking unit responsive to the electromagnetic signals to electromagnetically track the endoscope within the anatomical region relative to a global reference, and the intraoperative calibration of the electromagnetic tracker and the endoscopic camera is a function of both the image registration between the preoperative scan image of a calibration site within the anatomical region and one or more endoscopic images of the calibration site within the anatomical region and a function of an electromagnetic registration between the global reference and the preoperative scan image.

A third form of the present invention is a surgical navigation method involving an execution of an intraoperative calibration of the electromagnetic tracker and the endoscopic camera as a function of an image registration between the preoperative scan image of a calibration site within the anatomical region and one or more endoscopic images of the calibration site within the anatomical region, and a display of an image integration of the preoperative scan image of the anatomical region and the endoscopic image(s) of the anatomical region derived from the image registration.

For purposes of the present invention, the term “endoscope” is broadly defined herein as any device having the ability to image from inside a body and the term “endoscopic” is broadly defined herein as a characterization of any image acquired from such device. Examples of an endoscope for purposes of the present invention include, but are not limited to, any type of scope, flexible or rigid (e.g., arthroscope, bronchoscope, choledochoscope, colonoscope, cystoscope, duodenoscope, gastroscope, hysteroscope, laparoscope, laryngoscope, neuroscope, otoscope, push enteroscope, rhino laryngoscope, sigmoidoscope, sinuscope, thorascope, etc.) and any device similar to a scope that is equipped with an image system (e.g., a nested cannula with imaging). The imaging is local, and surface images may be obtained optically with fiber optics, lenses, or miniaturized (e.g. CCD based) imaging systems.

Additionally, the term “generating” and any form thereof as used herein is broadly defined to encompass any technique presently or subsequently known in the art for creating, supplying, furnishing, obtaining, producing, forming, developing, evolving, modifying, transforming, altering or otherwise making available information (e.g., data, text, images, voice and video) for computer processing and memory storage/retrieval purposes, particularly image datasets and video frames, and the term “registration” and any form thereof as used herein is broadly defined to encompass any technique presently or subsequently known in the art for transforming different sets of coordinate data into one coordinate system.

Furthermore, the term “preoperative” as used herein is broadly defined to describe any activity occurring or related to a period or preparations before an intervention of an endoscope within a body during an endoscopic application, and the term “intraoperative” as used herein is broadly defined to describe as any activity occurring, carried out, or encountered in the course of an introduction of an endoscope within a body during an endoscopic application. Examples of an endoscopic application include, but are not limited to, an arthroscopy, a bronchoscopy, a colonscopy, a laparoscopy, and a brain endoscopy.

The foregoing forms and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

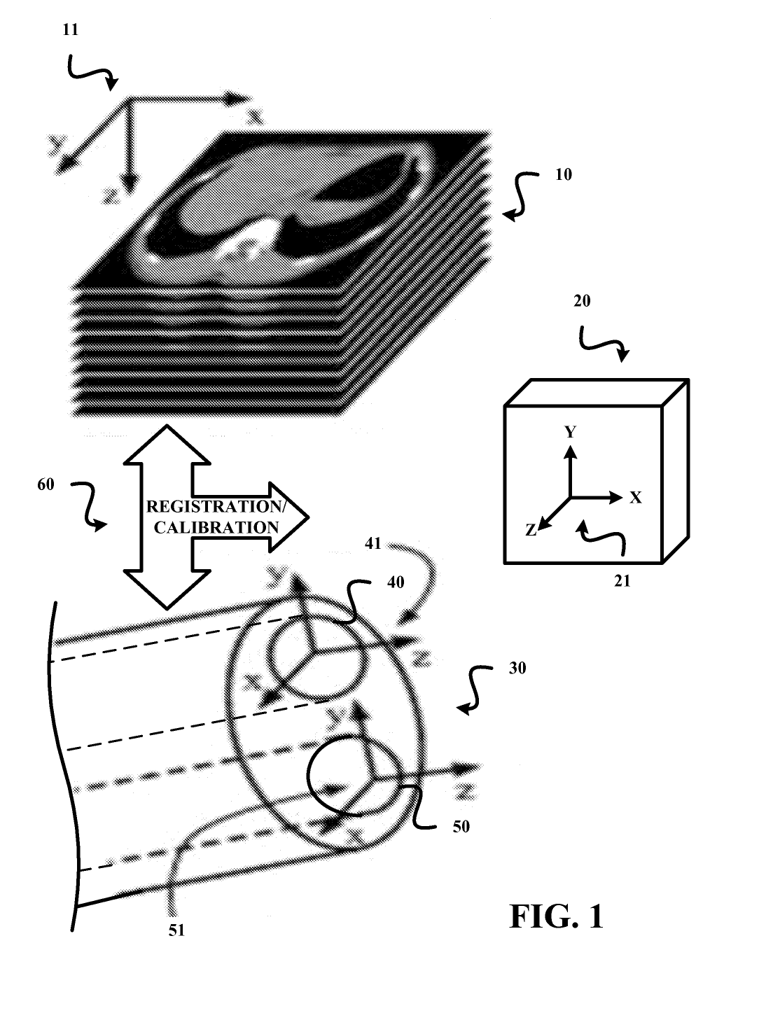

FIG. 1 illustrates en exemplary image registration in accordance with the present invention.

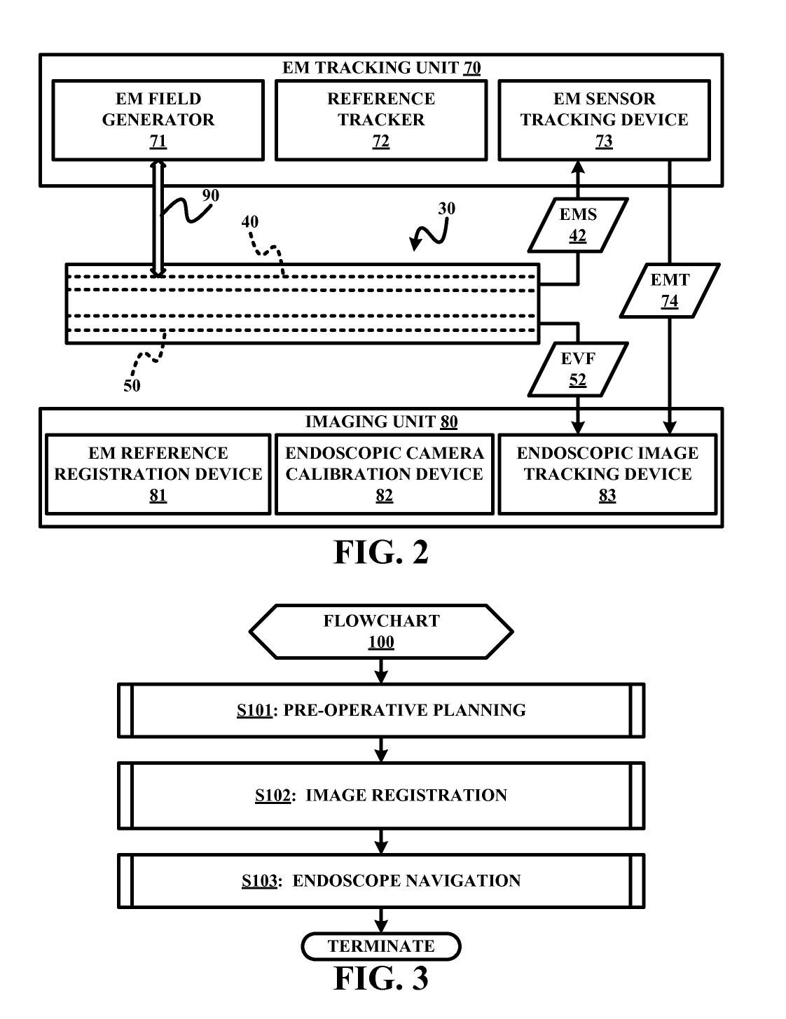

FIG. 2 illustrates an exemplary embodiment of a surgical navigation system in accordance with the present invention.

FIG. 3 illustrates a flowchart representative of an exemplary embodiment of an endoscopic surgical method in accordance with the present invention.

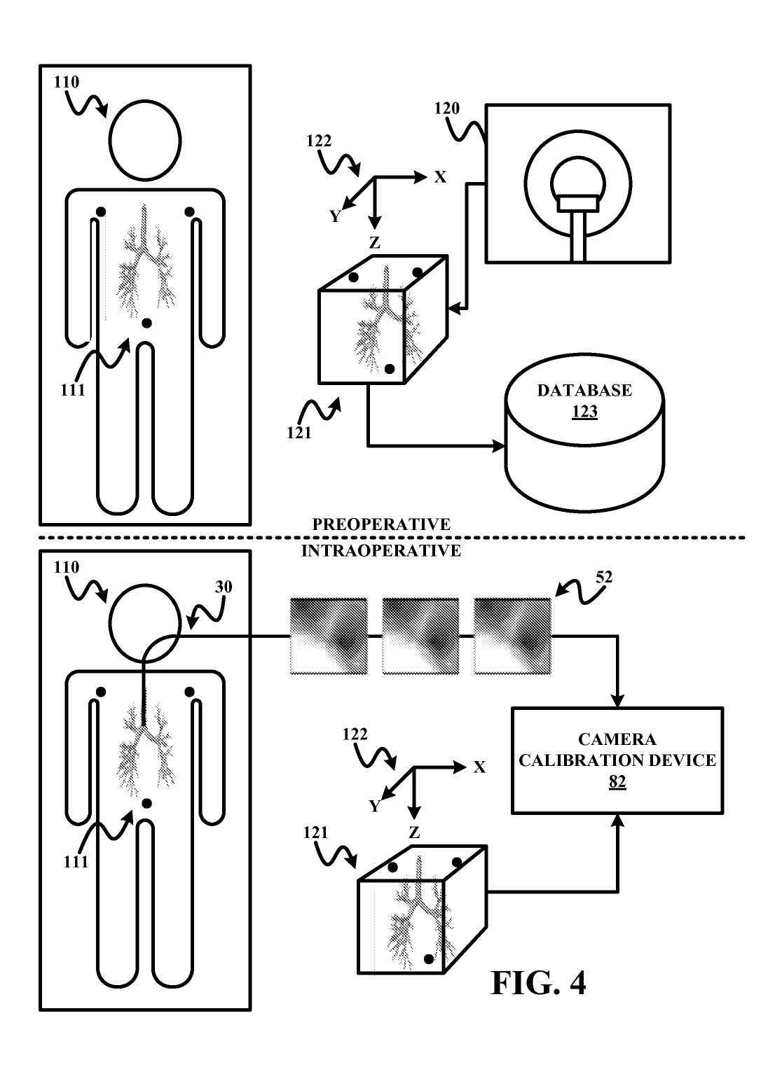

FIG. 4 illustrates an exemplary execution of the flowchart illustrated in FIG. 3.

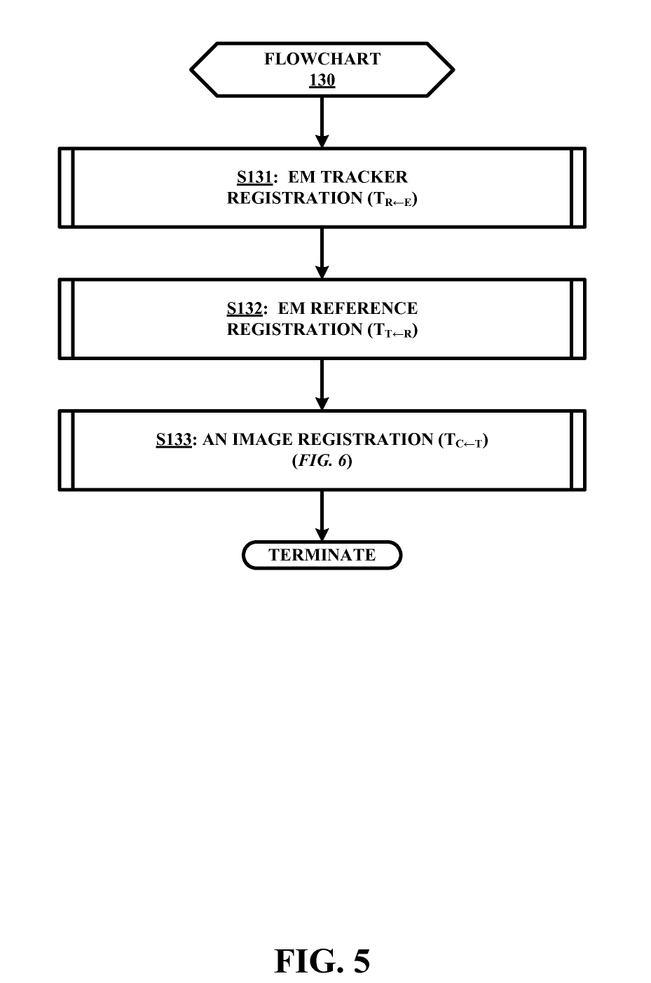

FIG. 5 illustrates a flowchart representative of an exemplary embodiment of an image registration method in accordance with the present invention.

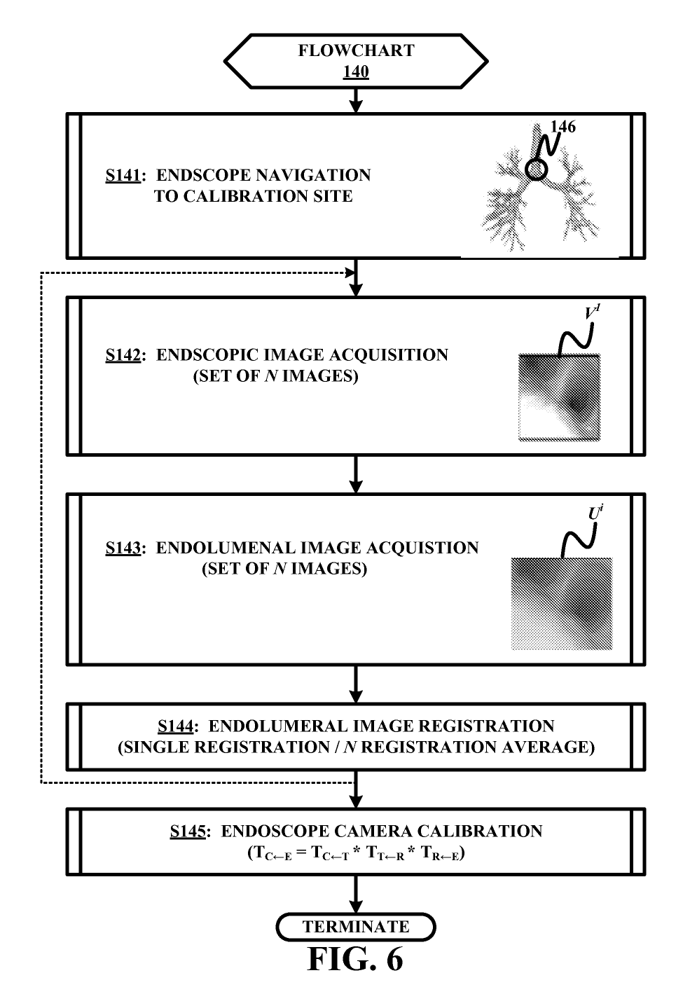

FIG. 6 illustrates a flowchart representative of an exemplary embodiment of an endoscopic camera calibration method in accordance with the present invention.

Referring to FIG. 1, the present invention is premised on a technique 60 for performing both an image registration and tracker/camera calibration during an intervention involving an endoscope 30. This registration/calibration technique 60 is grounded in the idea that an offset distance between a video frame from an endoscopic camera 50 and a tracking frame from a EM tracker 40 is reflected in a disparity in two-dimensional (“2D”) projection images between endoscopic images of an anatomical region (e.g., lungs) acquired from endoscopic camera 50 and a virtual fly-through of image frames of a preoperative scan image 10 of the anatomical region. As such, registration/calibration technique 60 has the capability to differentiate this spatial difference and the reconstructed spatial correspondence is used to estimate a calibration matrix between an EM tracking coordinate system 41 and an endoscopic camera coordinate system 51.

More particularly, intrinsic parameters and distortion parameters of endoscopic camera 50 are unchanging and as such, these parameters only require a one-time calibration process (e.g., a preoperative intrinsic calibration as known in the art). Thus, with EM tracker 40 being inserted into a working channel of endoscope 30, the only variable of all camera parameters is the extrinsic parameters, especially an offset transformation matrix T_{C←E} from EM tracker coordinate system 41 to camera coordinate system 51.

In practice, the present invention neither restricts or limits the manner by which registration/calibration technique 60 differentiates the disparity in the 2D projection images between endoscopic images of an anatomical region and a virtual fly-through of image frames of preoperative scan image 10 of the anatomical region.

In one embodiment, registration/calibration technique 60 involves the execution of the following equation [1]:

T_{C←E}=(T_{C←T})*(T_{T←R})*(T_{R←E}) [1]

where T_{R←E} is a transformation matrix as known in the art from EM tracker coordinate system 41 to a global coordinate system 21 of global reference 20 (e.g., a reference tracker or a EM field generator having a fixed location during the endoscopic surgical procedure),

where T_{T∂R} is a transformation matrix as known in the art from global coordinate system 21 of global reference 20 to scan image coordinate system 11 of preoperative scan image 10,

where T_{C←T} is a transformation matrix as taught by the present invention from scan image coordinate system 11 of preoperative scan image 10 to camera coordinate system 51 of endoscopic camera 50, and

where T_{C←E} is the desired rigid transformation from EM tracking coordinate system 41 of EM tracker 40 to camera coordinate system 51 of endoscopic camera 50.

An execution of equation [1] results in an image registration of the endoscopic images and preoperative scan image 10 for display to enable a surgeon to visually navigate the tip of endoscope 30 to a surgical site within the anatomical region.

FIG. 2 illustrates an endoscopic navigation system as an exemplary embodiment for implementing registration/calibration technique 60. To this end, endoscopic navigation system employs endoscope 30 and an EM tracking unit 70 having an EM field generator 71, a reference tracker 72 and an EM sensor tracking device 73.

As shown in FIG. 2, endoscope 30 includes EM tracker 40 inserted within a working channel of endoscope 30 and endoscopic camera 50 inserted within an imaging channel of endoscope 30. In practice, EM tracker 40 may have any configuration of EM sensors suitable for a magnetic interaction 90 with EM field generator 71 and for a generation of EM sensing data (“EMS”) 42 representative of magnetic interaction 90. For example, the EM sensors may have six (6) degrees of freedom (DOF).

Further, in practice, EM sensor tracking device 73 executes any known method for generating EM tracking data (“EMT”) 74 derived via any known registration of endoscope tracker 40 relative to EM field generator 71 or reference tracking device 72, whichever has a fixed location relative to the anatomical region within the global coordinate system.

The endoscopic navigation system further employs an endoscope imaging unit 80 having an EM reference registration device 81, an endoscopic camera calibration device 82 and an endoscopic image tracking device 83. EM tracker registration device 81 is broadly defined herein as any device structurally configured for executing any known registration of EM tracker 40 to a preoperative scan image of an anatomical region (e.g., preoperative scan image 10 of FIG. 1).

Endoscopic camera calibration device 82 is broadly defined herein as any device structurally configured for executing a registration of a preoperative scan image of an anatomical region to endoscopic images of the anatomical region in accordance with an endoscopic camera calibration method of the present invention as will be further explained in connection with the description of FIGS. 5 and 6.

Endoscopic image tracking device 83 is broadly defined herein as any device structurally configured for generating a display of a real-time tracking of endoscope 30 within the preoperative scan image based on the image registration between the endoscopic images and the preoperative scan image achieved by endoscopic camera calibration device 82.

A flowchart 100 representative of an endoscopic surgical method of the present invention as shown in FIG. 3 will now be described herein to facilitate a further understanding the endoscopic surgical navigation system of FIG. 2.

Referring to FIG. 3, a stage S101 of flowchart 100 encompasses a preoperative planning of the endoscopic surgery. For example, as shown in FIG. 4, the preoperative planning may involve a CT scanning machine 120 being operated to generate a preoperative scan image 121 of a bronchial tree of a patient 110. A set of fiducials 111 are captured in the preoperative scan image 121, which is stored in a database 123 to facilitate a subsequent EM registration of a global reference to preoperative scan image 121. A surgeon may use preoperative scan image 121 to identify a target site within the bronchial tree of patient 110 for delivery of a therapeutic agent via a working channel of endoscope 30.

Referring back to FIG. 3, a stage S102 of flowchart 100 encompasses an image registration of preoperative scan image 121 to endoscopic images generated from an endoscopic intervention. For example, as shown in FIG. 4, endoscope 30 is introduced into the bronchial tree of patient 110 whereby endoscopic images 52 of the bronchial tree are generated by endoscopic camera 50 (FIGS. 1 and 2). The image registration involves endoscopic camera calibration device 82 computing a transformation matrix T_{C←T} of the coordinate system 122 of preoperative image scan 121 to a coordinate system 51 (FIG. 1) of endoscopic camera 50.

In one embodiment, a flowchart 130 representative of an image registration method of the present invention as shown in FIG. 5 is executed during stage S102 of flowchart 100.

Referring to FIG. 5, a stage S131 of flowchart 130 encompasses an EM tracker registration involving a known computation by EM sensor tracking device 73 (FIG. 2) of transformation matrix T_{R←E} from EM tracker coordinate system 41 (FIG. 1) to a global coordinate system 21 (FIG. 1) of global reference 20.

A stage S132 of flowchart 130 encompasses an EM reference registration involving a known computation by EM reference registration device 81 (FIG. 2) of transformation matrix T_{T∂R} from global coordinate system 21 of global reference 20 to scan image coordinate system 122 of preoperative scan image 121 (FIG. 3). In particular, this EM reference registration may be achieved by a known closed form solution via a fiducial based method.

A stage S133 of flowchart 130 encompasses an image registration involving a computation by camera calibration device 82 of a transformation matrix T_{C←T} as taught by the present invention from scan image coordinate system 122 of preoperative scan image 120 to camera coordinate system 51 of endoscopic camera 50 (FIG. 1). This image registration includes a camera calibration involving a computation of an unknown transformation matrix T_{C←E} from EM tracker coordinate system 41 of EM tracker 40 to camera coordinate system 51 of endoscopic camera 50.

In one embodiment of stage S133, a flowchart 140 representative of a camera calibration method of the present invention as shown in FIG. 6 is executed by camera calibration device 82 for computing transformation matrix T_{C←E} from EM tracker coordinate system 41 of EM tracker 40 to camera coordinate system 51 of endoscopic camera 50.

Referring to FIG. 6, a stage S141 of flowchart 140 encompasses a navigation of an endoscope for imaging a calibration site within the anatomical region. The calibration site is a user defined location within the anatomical region that remains relatively stable during the calibration process. For example, the calibration site may be a main carina 146 of a bronchial tree as shown in FIG. 6. Specifically, research indicates main carina 146 remains relatively stable during respiratory cycles of the bronchial tree. As such, endoscope 30 may be navigated by surgeon for imaging carina 146 to perform the camera calibration computation of stages S142-S145.

Specifically, stages S142-S144 of flowchart 140 respectively encompass an acquisition of a video frame Vⁱ of endoscopic image of the calibration site, a rendering of an scan frame Uⁱ of an endoluminal image of the calibration site, and an image registration between scan frame Uⁱ of an endoluminal image of the calibration site and the video frame Vⁱ of the calibration site to identify the camera poses in the pre-operative scan space Tⁱ_{T←C}. The endoscopic image acquisition of stage S142 involves an EM tracker reading P_{R<-E}ⁱ to obtain a pose of endoscope 30 associated with the endoscopic image acquisition. The endoluminal image acquisition of stage 143 involves a virtual endoscopic flythrough of the preoperative scan image of the anatomical region to thereby obtain a visual match of an endoscopic view of the calibration site as shown in a scan frame Uⁱ of the preoperative scan image with the endoscopic image of the calibration site as shown in video frame Vⁱ. The endoluminal image registration of stage S144 involves a computation 4×4 transformation matrix Tⁱ_{C←T} as an inverse of matrix Tⁱ_{T←C} hereby the camera viewing pose is expressed as M=[RₓTₓ;0 1], where Rₓ is the corresponding Euler 3×3 rotation matrix of the 3D translation vector and Tₓ is the 3D translation vector.

Stages S142-S144 may be executed a single time whereby a stage S145 of flowchart encompasses an execution of equation [1]: T_{C←E}=(T_{C←T})*(T_{T←R})*(T_{R←E}) to thereby obtain the transformation matrix T_{C←E}.

Alternatively, stages S142-S144 may be executed as a loop for a set of N image registrations, wherein N≧2. For this loop embodiment, the transformation matrixes T_{C-T} computed during each execution of stage S144 are averaged prior to the endoscopic camera calibration computation of stage S145.

In practice, N=6 may be utilized as a sufficient number of image registrations for an accurate computation of the camera calibration.

Furthermore, in practice, a known motion compensation algorithm (e.g., respiratory gating or four-dimensional modeling) may be utilized to compensate for any respiratory motion that my degrade the computation of the camera calibration.

Referring to back to FIG. 2, upon the image registration of the endoscopic images and the preoperative image scan, a stage S103 of flowchart 100 encompasses a display of the integrated images as known in the art to facilitate a navigation of the endoscope to a surgical site within the anatomical region.

Referring to FIGS. 1-6, those having ordinary skill in the art will appreciate the various benefits of the present invention including, but not limited to, an intraoperative camera calibration that provides a sufficiently accurate image registration for navigating an endoscope to a surgical site whereby the EM tracker may be removed from a working channel of the endoscope and a surgical tool inserted into the working channel for performing the needed procedure at the surgical site.

While various embodiments of the present invention have been illustrated and described, it will be understood by those skilled in the art that the methods and the system as described herein are illustrative, and various changes and modifications may be made and equivalents may be substituted for elements thereof without departing from the true scope of the present invention. In addition, many modifications may be made to adapt the teachings of the present invention without departing from its central scope. Therefore, it is intended that the present invention not be limited to the particular embodiments disclosed as the best mode contemplated for carrying out the present invention, but that the present invention include all embodiments falling within the scope of the appended claims.

1. A surgical navigation system, comprising:
an endoscope (30) including
an electromagnetic tracker (40) within a working channel of the endoscope (30) for generating electromagnetic sensing signals indicative of at least one pose of the endoscope (30) within an anatomical region, and
an endoscopic camera (50) within an imaging channel of the endoscope (30) for generating endoscopic images of the anatomical region; and
an imaging unit (80) operable to generate an intraoperative calibration of the electromagnetic tracker (40) and the endoscopic camera (50) as a function of an image registration between a preoperative scan image of a calibration site within the anatomical region and at least one endoscopic image of the calibration site within the anatomical region.

2. The surgical navigation system of claim 1, wherein the image registration includes:
navigating the endoscope (30) to a first pose within the anatomical region relative to the calibration site;
acquiring a first endoscopic image of the calibration site corresponding to the first pose of the endoscope (30) within the anatomical region relative to the calibration site;
executing a virtual endoscopic flythrough of the preoperative scan image to the first pose of the endoscope (30) within the anatomical region relative to the calibration site;
acquiring a first endoluminal image of the calibration site corresponding to the first pose of the endoscope (30) within the anatomical region relative to the calibration site; and
registering the first endoluminal image of the calibration site and the first endoscopic image of the calibration site including a computation of a first image transformation matrix (T_{C←T}).

3. The surgical navigation system of claim 2, wherein the anatomical region is a bronchial tree and the calibration site is a main carina.

4. The surgical navigation system of claim 2, wherein the intraoperative calibration further includes:
computing a calibration transformation matrix (T_{C←E}) as a function of the first image transformation matrix (T_{C←T}), an electromagnetic tracker transformation matrix (T_{R←E}) from the endoscope (30) tracker to a global reference, and an electromagnetic reference transformation matrix (T_{T←R}) from the global reference to the preoperative scan image of the anatomical region.

5. The surgical navigation system of claim 2, wherein the image registration includes:
navigating the endoscope (30) to a second pose within the anatomical region relative to the calibration site;
acquiring a second endoscopic image of the calibration site corresponding to the second pose of the endoscope (30) within the anatomical region relative to the calibration site;
executing a virtual endoscopic flythrough of the preoperative scan image to the second pose of the endoscope (30) within the anatomical region relative to the calibration site;
acquiring a second endoluminal image of the calibration site corresponding to the second pose of the endoscope (30) within the anatomical region relative to the calibration site; and
registering the second endoluminal image of the calibration site and the second endoscopic image of the calibration site including a computation of a second image transformation matrix (T_{C←T}).

6. The surgical navigation system of claim 5, wherein the intraoperative calibration includes:
averaging the first image transformation matrix (T_{C←T}) and the second image transformation matrix (T_{C←T}); and
computing a calibration transformation matrix (T_{C←E}) as a function of the averaged image transformation matrix (T_{C←T}), an electromagnetic tracker transformation matrix (T_{R←E}) from the endoscope (30) tracker to a global reference, and an electromagnetic reference transformation matrix (T_{T←R}) from the global reference to the preoperative scan image of the anatomical region.

7. The surgical navigation system of claim 1, wherein the imaging unit (80) is further operable to display an image integration of the preoperative scan image of the anatomical region and the at least one endoscopic image of the anatomical region derived from the image registration.

8. The surgical navigation system of claim 7, wherein:
the endoscope (30) is operable to be navigated to a surgical pose within the anatomical region relative to a surgical site as displayed by the image integration;
the electromagnetic tracker (40) is operable to be removed from the working channel subsequent to the endoscope (30) being navigated to the surgical pose; and
a surgical instrument is operable to be inserted within the working channel subsequent to a removal of the electromagnetic tracker (40) from the working channel.

9. A surgical navigation system, comprising:
an endoscope (30) including
an electromagnetic tracker (40) within a working channel of the endoscope (30) for generating electromagnetic sensing signals indicative of at least one pose of the endoscope (30) within an anatomical region, and
an endoscopic camera (50) within an imaging channel of the endoscope (30) for generating endoscopic images of the anatomical region;
an electromagnetic tracking unit responsive to the electromagnetic signals to electromagnetic track the endoscope (30) within the anatomical region relative to a global reference; and
an imaging unit (80) operable to execute an intraoperative calibration of the electromagnetic tracker (40) and the endoscopic camera (50) as a function of an image registration between a preoperative scan image of a calibration site within the anatomical region and at least one endoscopic image of the calibration site within the anatomical region and as a function of an electromagnetic registration of the global reference and the preoperative scan image.

10. The surgical navigation system of claim 9, wherein the image registration includes:
navigating the endoscope (30) to a first pose within the anatomical region relative to the calibration site;
acquiring a first endoscopic image of the calibration site corresponding to the first pose of the endoscope (30) within the anatomical region relative to the calibration site;
executing a virtual endoscopic flythrough of the preoperative scan image to the first pose of the endoscope (30) within the anatomical region relative to the calibration site;
acquiring a first endoluminal image of the calibration site corresponding to the first pose of the endoscope (30) within the anatomical region relative to the calibration site; and
registering the first image of the calibration site and the first endoscopic image of the calibration site including a computation of a first image transformation matrix (T_{C←T}).

11. The surgical navigation system of claim 10, wherein the intraoperative calibration includes:
computing a calibration transformation matrix (T_{C←E}) as a function of the first image transformation matrix (T_{C←T}), an electromagnetic tracker (40) transformation matrix (T_{R←E}) from the endoscope (30) tracker to the global reference, and an electromagnetic reference transformation matrix (T_{T←R}) from the global reference to the preoperative scan image of the anatomical region.

12. The surgical navigation system of claim 10, wherein the image registration further includes:
navigating the endoscope (30) to a second pose within the anatomical region relative to the calibration site;
acquiring a second endoscopic image of the calibration site corresponding to the second pose of the endoscope (30) within the anatomical region relative to the calibration site;
executing a virtual endoscopic flythrough of the preoperative scan image to the second pose of the endoscope (30) within the anatomical region relative to the calibration site;
acquiring a second endoluminal image of the calibration site corresponding to the second pose of the endoscope (30) within the anatomical region relative to the calibration site; and
registering the second endoluminal image of the calibration site and the second endoscopic image of the calibration site including a computation of a second image transformation matrix (T_{C←T}).

13. The surgical navigation system of claim 12, wherein the intraoperative calibration includes:
averaging the first image transformation matrix (T_{C←T}) and the second image transformation matrix (T_{C←T}); and
computing a calibration transformation matrix (T_{C←E}) as a function of the averaged image transformation matrix (T_{C←T}), an electromagnetic tracker (40) transformation matrix (T_{R←E}) from the endoscope (30) tracker to a global reference, and an electromagnetic reference transformation matrix (T_{T←R}) from the global reference to the preoperative scan image of the anatomical region.

14. The surgical navigation system of claim 1, wherein the imaging unit (80) is further operable to display an image integration of the preoperative scan image of the anatomical region and the at least one endoscopic image of the anatomical region derived from the image registration.

15. The surgical navigation system of claim 14, wherein:
the endoscope (30) is operable to be navigated to a surgical pose within the anatomical region relative to a surgical site as displayed by the image integration;
the electromagnetic tracker (40) is operable to be removed from the working channel subsequent to the endoscope (30) being navigated to the surgical pose; and
a surgical instrument is operable to be inserted within the working channel subsequent to a removal of the electromagnetic tracker (40) from the working channel.

16. A surgical navigation method, comprising:
executing an intraoperative calibration of an electromagnetic tracker (40) and an endoscopic camera (50) as a function of an image registration of a preoperative scan image of a calibration site within an anatomical region to at least one endoscopic image of the calibration site within the anatomical region; and
displaying an image integration of the preoperative scan image of the anatomical region and the at least one endoscopic image of the anatomical region derived from the image registration.

17. The surgical navigation method of claim 16, wherein the image registration includes:
navigating the endoscope (30) to a first pose within the anatomical region relative to the calibration site;
acquiring a first endoscopic image of the calibration site corresponding to the first pose of the endoscope (30) within the anatomical region relative to the calibration site;
executing a virtual endoscopic flythrough of the preoperative scan image to the first pose of the endoscope (30) within the anatomical region relative to the calibration site;
acquiring a first endoluminal image of the calibration site corresponding to the first pose of the endoscope (30) within the anatomical region relative to the calibration site; and
registering the first endoluminal image of the calibration site and the first endoscopic image of the calibration site including a computation of a first image transformation matrix (T_{C←T}).

18. The surgical navigation method of claim 17, wherein the intraoperative calibration includes:
computing a calibration transformation matrix (T_{C←E}) as a function of the first image transformation matrix (T_{C←T}), an electromagnetic tracker (40) transformation matrix (T_{R←E}) from the endoscope (30) tracker to the global reference, and an electromagnetic reference transformation matrix (T_{T←R}) from the global reference to the preoperative scan image of the anatomical region.

19. The surgical navigation method of claim 17, wherein the image registration further includes:
navigating the endoscope (30) to a second pose within the anatomical region relative to the calibration site;
acquiring a second endoscopic image of the calibration site corresponding to the second pose of the endoscope (30) within the anatomical region relative to the calibration site;
executing a virtual endoscopic flythrough of the preoperative scan image to the second pose of the endoscope (30) within the anatomical region relative to the calibration site;
acquiring a second endoluminal image of the calibration site corresponding to the second pose of the endoscope (30) within the anatomical region relative to the calibration site; and
registering the second endoluminal image of the calibration site and the second endoscopic image of the calibration site including a computation of a second image transformation matrix (T_{C←T}).

20. The surgical navigation method of claim 19, wherein the intraoperative calibration includes:
averaging the first image transformation matrix (T_{C←T}) and the second image transformation matrix (T_{C←T}); and
computing a calibration transformation matrix (T_{C←E}) as a function of the averaged image transformation matrix (T_{C←T}), an electromagnetic tracker (40) transformation matrix (T_{R←E}) from the endoscope (30) tracker to a global reference, and an electromagnetic reference transformation matrix (T_{T←R}) from the global reference to the preoperative scan image of the anatomical region.
